(19) United States
(12) Patent Application Publication
(10) Pub. No.: US 2007/0027097 A1
(43) Pub. Date: Feb. 1, 2007

(54) Small interfering RNA with improved activity

(76) Inventor: David L. Lewis, Madison, WI (US)

Correspondence Address: MIRUS CORPORATION, 505 SOUTH ROSA RD, MADISON, WI, 53719, US

(21) Appl. No.: 11/192,354

(22) Filed: Jul. 28, 2005

(51) Int. Cl. A61K 48/00; C07H 21/02

(52) U.S. Cl. 514/44; 536/23.1

(57) ABSTRACT

Chemically modified small interfering RNAs are described. Combinations of 2′-hydroxyl substitutions on the nucleotide riboses are shown to increase the longevity and extent of target gene knockdown in mammalian cells.

### CROSS-REFERENCE TO RELATED APPLICATIONS

This application claims the benefit of U.S. Provisional Application No. 60/581,068 filed Jun. 18, 2004.

### FIELD OF THE INVENTION

Use of chemically modified small interfering RNAs to increase the longevity and extent of target gene knockdown in mammalian cells in culture and in vivo.

### BACKGROUND OF THE INVENTION

Recently, there has been a great deal of research interest in the delivery of RNA oligonucleotides to cells due to the discovery of RNA interference (RNAi). RNAi interference results in the knockdown of protein production within cells, via the interference of the small interfering RNA (siRNA) with the mRNA involved in protein production. This interference curtails gene expression. The delivery of small double stranded RNAs (small interfering RNAs, or siRNAs, and microRNAs) to cells can result in a greater than 80% knockdown of endogenous gene expression levels within the cell. Additionally, through the use of specific siRNAs, gene knockdown can be accomplished without inhibiting the expression of non-targeted genes.

The inhibitory effects of siRNA are transient in mammalian cells, possibly because of susceptibility of the siRNA to degradation by nucleases. The use of chemical modifications to enhance nuclease resistance would be predicted to increase the longevity of the siRNA and in turn, increase the persistence of target gene knockdown. However, most modifications to siRNA negatively impact siRNA activity. For example, substitution of the 2′-OH (hydroxyl) group with 2′-OCH₃ (methoxy) on every nucleotide in the sense or antisense strands of the siRNA has a severely negative impact on siRNA activity (Chiu 2003, Braasch 2003, Czaudema 2003). Some activity is retained if substitution is limited to stretches of five nucleotides, the substitutions are present only at the 5′ and 3′ ends or only every other nucleotide contains a substitution, depending on the register of the substituted nucleotides (Czaudema 2003). Substitution with deoxynucleotides at every nucleotide position on either the sense or antisense strands also has a negative impact on siRNA activity (Chiu 2003, Holen 2003). In contrast, substitution of the 2′-OH group on pyrimidines of either or both strands of the siRNA with 2′-F has a negligible effect on siRNA activity (Capodici 2002, Chiu 2003, Harborth 2003). The activity of siRNA containing 2′-F nucleotides at all positions has not been reported.

In addition to the aforementioned substitutions, modifications of the 5′ and 3′ terminal nucleotides of both strands of the siRNA and their impact on siRNA activity have also been reported. Modifications at these positions are well tolerated, except when present on the 5′ position of the antisense strand (Chiu 2002, Martinez 2002, Harborth 2003). Modifications at this position likely disrupt binding of the guide strand of the siRNA to components of the dsRNA-induced silencing complex (RISC, Ma 2005). There is a need to identify siRNA analogs that retain full activity of the siRNA and increase the persistence of target gene knockdown.

### SUMMARY OF THE INVENTION

In a preferred embodiment, methods and compositions are provided that increase the longevity and extent of gene knockdown in mammals after delivery of double-stranded RNA molecules. Modifications of the ribose sugar and phosphate backbone of the double stranded RNA molecule are described. Delivery of the modified dsRNA molecules to non-embryonic mammals results in higher levels of target gene knockdown for longer periods of time compared to unmodified dsRNA molecules.

In a preferred embodiment, we describe modified siRNAs that exhibit prolonged gene knockdown activity. The modification comprises substitution of the 2′ hydroxyl group on the ribose sugars of the siRNA. Preferred modifications comprise 2′-methoxy groups (—OCH₃ or —OMe) on purine ribonucleotides and 2′-fluoro groups (—F) on pyrimidine ribonucleotides. The siRNA may contain modified bases on the sense strand, antisense strand, or both strands. A preferred modified siRNA contains modified bases in only the sense strand. In another embodiment, the modified siRNA contains 2′-F substitutions at every ribonucleotide position in either the sense strand, the antisense strand, or both strands. In another preferred embodiment, the siRNA molecules contain two terminal thymidine deoxyribonucleotides connected though a 3′ phosphate to 3′ phosphate linkage.

Further objects, features, and advantages of the invention will be apparent from the following detailed description when taken in conjunction with the accompanying drawings.

### DETAILED DESCRIPTION

The present invention provides compositions for RNA interference and methods of use thereof. In particular, the invention pertains to compounds effective at increasing or prolonging RNA interference induced by siRNA in a cell or organism. Modified small interfering RNAs (siRNAs) are described. Delivery of the modified siRNAs to cells results in more persistent inhibition of target gene expression, more efficient target gene knock down, or both.

An siRNA comprises a polynucleotide or polynucleotide analog comprising a sequence whose presence or expression in a cell causes the degradation of or inhibits the function or translation of a specific cellular RNA, usually an mRNA, in a sequence-specific manner. Inhibition of RNA can thus effectively inhibit expression of a gene from which the RNA is transcribed. SiRNA, when delivered to mammalian cells, inhibits gene expression through RNA interference (RNAi). For the purposes of this invention, siRNA includes siRNA, microRNA (miRNA), small hairpin RNA (shRNA), short double strand RNA or other nucleic acids that induce RNAi. SiRNA comprises a double stranded structure typically containing 15-50 base pairs and preferably 19-25 base pairs and having a nucleotide sequence identical or nearly identical to an expressed target gene or RNA within the cell. An siRNA may be composed of two annealed polynucleotides or a single polynucleotide that forms a hairpin structure. MicroRNAs (miRNAs) are small noncoding polynucleotides, about 22 nucleotides long, that direct destruction or translational repression of their mRNA targets. SiRNAs may contain ribonucleotides, deoxyribonucleotides, synthetic nucleotides, or any suitable combination such that the target RNA and/or gene is inhibited. Inhibition of gene expression refers to a decrease in the level of protein and/or mRNA product from a target gene.

The siRNAs of the present invention contain substitutions at the 2′ carbons of the nucleotide riboses in the nucleotide backbone. In one embodiment, purine ribonucleotides of the siRNA are modified to contain 2′-OMe substitutions and pyrimidine ribonucleotides of the siRNA are modified to contain 2′-F substitutions. In another embodiment both purine and pyrimidine ribonucleotides of the siRNA are modified to contain 2′-F substitutions. In one embodiment, the modified nucleotides are present only in the sense strand. In another embodiment, the modified nucleotides are present only in the antisense strand. In another embodiment, the modified nucleotides are present in both the sense and antisense strand. In a preferred embodiment, the modified siRNA contains substitutions at every position or nearly every position of the sense strand, antisense strand, or both. Thus, in one embodiment, the modified siRNA of the present invention comprise double strand ribonucleotides wherein at least one of the strands is composed essentially of 2′-OMe purine ribonucleotides and 2′-F pyrimidine ribonucleotides. In another embodiment, the siRNA of the present invention comprise double strand ribonucleotides wherein at least one of the strands is composed essentially of 2′-F pyrimidine ribonucleotides. Modification of siRNA results in increased potency and longevity of target gene knockdown.

The modified siRNA of the present invention may have a 3′ overhang of about 1 to about 6 nucleotides in length. More preferably, the 3′ overhangs are 1-3 nucleotides in length. The length of the overhangs may be the same or different for each strand. In order to further enhance the stability of the siRNA, the 3′ overhangs can be stabilized against degradation. The 3′ terminal nucleotide of the oligo may be connected to the adjacent (penultimate) nucleotide through a 3′-PO₄-3′ linkage (e.g., an inverted nucleotide). The oligonucleotide may also have a 3′ abasic nucleotide or inverted 3′ abasic nucleotide. The 3′ overhangs may or may not have 2′-OCH₃ or 2′-F substitutions or they may be deoxyribonucleotides. Other 5′ or 3′ modifications are permissible provided they do not inactivate the siRNA.

The modified siRNAs of the present invention may also be in the form of a hairpin structure (hairpin siRNA). For hairpin siRNAs, the sense sequences and antisense sequences are present in a single molecule connected by a loop of about 4 to about 30 nucleotides and more preferably from about 4 to about 9 nucleotides. The sugars, phosphate linkages or bases of the loop nucleotides may be modified. Examples of making and using such hairpin RNAs for gene silencing in mammalian cells are described in, for example, Paddison et al. 2002, McCaffrey 2002, McManus et al. 2002, Yu et al. 2002.

The sense strand or sequence comprises a nucleotide sequence that is identical or substantially identical to a nucleotide sequence in the target mRNA. The antisense strand or sequence comprises a nucleotide sequence that is complementary or substantially complementary to the sense strand sequence.

The siRNA may include one or more modified phosphate linkages. For example, the phosphodiester linkages of natural RNA may be modified to include at least one of a nitrogen or sulfur heteroatom (phosphoimidate or phosphothioester linkages). The phosphodiester linkages may be modified within the sense strand, within the antisense strand, or within the sense and antisense strands.

Effective siRNA sequences are readily identified through methods readily known in the art. A number of rules or guidelines and algorithms have been developed for predicting effective siRNA sequences: Reynolds et al. 2004, Khvorova et al. 2003, Schwarz et al. 2003, Ui-Tei et al. 20043, Heale et al. 2005, Chalk et al. 2004, Amarzguioui et al. 2004. SiRNA sequences can be designed according to convention, including tolerance of mismatches between the sense sequence and the antisense sequence of the siRNA and between the siRNA and the target sequence. The effectiveness of any given sequence or modification thereof is readily determined using assay systems known in the art and as described below in the examples. Any system in which RNAi activity can be detected can be used to test the activity of a candidate siRNA or modified siRNA.

The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

The effectiveness of the siRNAs of the present invention are not limited to any particular method of delivery to cells. The process of delivering a nucleic acid to a cell has been commonly termed transfection or the process of transfecting and has also been termed transformation. The siRNAs of the present invention may therefore be delivered using any known in vivo or in vitro delivery system that is effective in delivering small polynucleotides. Known delivery systems include, but are not limited to: intravascular injection, hydrodynamic injection, viral vectors, electroporation, biolistic methods, and non-viral vectors. Non-viral vectors include transfection reagents such as polycations, cationic and non-cationic lipids, and amphipathic compounds. For delivery to a cell in vivo, the modified siRNA of the present invention may be in a pharmaceutically acceptable carrier. The siRNAs may be associated or linked with other compounds that aid in delivery. The siRNA may also be labeled to allow detection of the siRNA in the cell.

Modified siRNA can be delivered to cells in vivo, in situ, ex vivo, or in vitro. In vitro cells include, but are not limited to, cell lines that can be obtained from American Type Culture Collection (Bethesda) such as: 3T3 (mouse fibroblast) cells, Rat1 (rat fibroblast) cells, CHO (Chinese hamster ovary) cells, CV-1 (monkey kidney) cells, COS (monkey kidney) cells, 293 (human embryonic kidney) cells, HeLa (human cervical carcinoma) cells, HepG2 (human hepatocytes) cells, Sf9 (insect ovarian epithelial) cells and the like.

In one embodiment, the modified siRNA may be delivered to a mammalian cell in vivo for the treatment of a disease or infection. The siRNA may target an endogenous gene or a gene of an infectious agent such as a virus. The inhibitor may reduce or block microbe production, virulence, or both. Delivery of the inhibitor may delay progression of disease until endogenous immune protection can be acquired or other treatment provided. Viral genes involved in transcription, replication, virion assembly, immature viral membrane formation, extracellular enveloped virus formation, early genes, intermediate genes, late genes, and virulence genes may be targeted. The siRNA may also decrease expression of an endogenous host gene to reduce virulence of the pathogen. The inhibitor may be delivered to a cell in a mammal to reduce expression of a cellular receptor. For example, the lethality of Anthrax is primarily mediated by a secreted tripartite toxin which requires the mammalian anthrax toxin receptor (ATR) for cellular entry. Reducing expression of ATR may decrease Anthrax toxicity. Receptors to which pathogens bind may also be targeted. Endogenous genes include dysfunctional genes, such as dominant negative genes that cause disease or cancer. In one embodiment, combinations of modified siRNAs targeted to the same or different genes may be delivered a mammalian cell.

The siRNA may be delivered to a mammal suffering from a condition, or may be delivered prior to clinical manifestation of the unwanted condition to protect the mammal against developing the unwanted condition.

In one embodiment, the modified siRNA may be delivered to a mammalian cell in vivo to modulate immune response. Since host immune response is responsible for the toxicity of some infectious agents, reducing this response may increase the survival of an infected mammal. Also, inhibition of immune response is beneficial for a number of other therapeutic purposes, including gene therapy, where immune reaction often greatly limits transgene expression, organ transplantation, and autoimmune disorders.

In one embodiment, the modified siRNA may be delivered to a mammalian cell in vivo or in vitro for the purpose of facilitating pharmaceutical drug discovery or target validation. Specific inhibition of a target gene can aid in determining whether inhibition of a protein or gene has a significant phenotypic effect. Specific inhibition of a target gene can also be used to study the target gene's effect on the cell.

In one embodiment, the modified siRNA may be delivered to a mammalian cell in vivo or in vitro to study gene function, to facilitate analysis of gene expression profiles and proteomes or for biomedical investigation.

An effective amount of a modified siRNA to be delivered to a cell refers to an amount of the siRNA in a preparation which, when applied as part of a desired dosage regimen, provides a benefit according to clinically acceptable standards for the treatment or prophylaxis of a particular disorder. Similarly, an effective amount of a modified siRNA to be delivered to a cell for research purposes refers to the amount of siRNA which provides the desired level of gene inhibition without causing unacceptable non-specific effects.

The term polynucleotide, or nucleic acid or polynucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, β-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term polynucleotide includes deoxyribonucleic acid (DNA) and ribonucleic acid (RNA) and combinations of DNA, RNA and other natural and synthetic nucleotides.

A transfection reagent is a compound or compounds that bind(s) to or complex(es) with oligonucleotides and polynucleotides, and mediates their entry into cells. The transfection reagent also mediates the binding and internalization of oligonucleotides and polynucleotides into cells. Examples of transfection reagents include, but are not limited to, cationic lipids and liposomes, polyamines, calcium phosphate precipitates, histone proteins, polyethylenimine, and polylysine complexes. It has been shown that cationic proteins like histones and protamines, or synthetic cationic polymers like polylysine, polyarginine, polyornithine, DEAE dextran, polybrene, and polyethylenimine may be effective intracellular delivery agents, while small polycations like spermine are ineffective. Typically, the transfection reagent has a net positive charge that binds to the oligonucleotide's or polynucleotide's negative charge. The transfection reagent mediates binding of oligonucleotides and polynucleotides to cells via its positive charge (that binds to the cell membrane's negative charge) or via cell targeting signals that bind to receptors on or in the cell. For example, cationic liposomes or polylysine complexes have net positive charges that enable them to bind to DNA or RNA. Polyethylenimine, which facilitates gene transfer without additional treatments, probably disrupts endosomal function itself.

### EXAMPLES

1. SiRNAs: Six 21-mer oligonucleotides (3 sense and 3 antisense) were synthesized for each of three target sequences in the Secreted Alkaline Phosphatase (SEAP) gene (Accession number U89937), SEAP-360, SEAP1116 and SEAP-1296. One sense strand and one antisense strand 21-mer oligonucleotide were synthesized for the target sequence (GL3-153) in the Photinus pyralis luciferase gene (GL3; Accession number U47296). The luciferase-specific siRNA was used as a negative control.

A) SEAP-360: Target sequence is position 360-380 of the SEAP open reading frame:

| | |
|---|---|
| CAAGGGCAACTTCCAGACCAT. | (SEQ ID 1) |

Unmodified SEAP-360 RNA oligonucleotides (SEAP-360-U oligonucleotides):

| | |
|---|---|
| SEAP-360-U(s): | |
| 5′ PO₄-AGGGCAACUUCCAGACCAUdTdT | (SEQ ID 2) |
| SEAP-360-U(as): | |
| 5′ PO₄-AUGGUCUGGAAGUUGCCCUdTdT | (SEQ ID 3) |

Modified SEAP-360 RNA oligonucleotides (SEAP-360-m/f oligonucleotides):

| | |
|---|---|
| SEAP-360-m/f(s): | |
| 5′ PO₄-ₘAₘGₘGₘG_{f}CₘAₘA_{f}C_{f}U_{f}U_{f}C_{f}CₘAₘGₘA_{f} | (SEQ ID 2) |
| C_{f}CₘA_{f}U_{d}T_{id}T | |
| SEAP-360-m/f(as): | |
| 5′ PO₄-ₘA_{f}UₘGₘG_{f}U_{f}C_{f}UₘGₘGₘAₘAₘG_{f}U_{f}UₘG_{f} | (SEQ ID 3) |
| C_{f}C_{f}C_{f}U_{d}T_{id}T | |

SEAP-360-f oligonucleotides:

| | |
|---|---|
| SEAP-360-f(s): | |
| 5′ PO₄-_{f}A_{f}G_{f}G_{f}G_{f}C_{f}A_{f}A_{f}C_{f}U_{f}U_{f}C_{f}C_{f}A_{f}G_{f}A_{f} | (SEQ ID 2) |
| C_{f}C_{f}A_{f}U_{d}T_{id}T | |
| SEAP-360-f(as): | |
| 5′ PO₄-_{f}A_{f}U_{f}G_{f}G_{f}U_{f}C_{f}U_{f}G_{f}G_{f}A_{f}A_{f}G_{f}U_{f}U_{f}G_{f} | (SEQ ID 3) |
| C_{f}C_{f}C_{f}U_{d}T_{id}T | |

B) SEAP-1116: Target sequence is position 1035-1055 of the SEAP open reading frame:

| | |
|---|---|
| GACTGAGACGATCATGTTCGA | (SEQ ID 4) |

Unmodified SEAP-1116 RNA oligonucleotides (SEAP-1116-U RNA oligonucleotides):

| | |
|---|---|
| SEAP-1116-U(s): | |
| 5′ PO₄-CUGAGACGAUCAUGUUCGAdTdT | (SEQ ID 5) |
| SEAP-1116-U(as): | |
| 5′ PO₄-UCGAACAUGAUCGUCUCAGdTdT | (SEQ ID 6) |

Modified SEAP-1116 RNA oligonucleotides (SEAP-1116-m/f oligonucleotides):

| | |
|---|---|
| SEAP-1116-m/f(s): | |
| 5′ PO₄-_{f}C_{f}UₘGₘAₘGₘA_{f}CₘGₘA_{f}U_{f}CₘA_{f}UₘG_{f}U_{f} | (SEQ ID 5) |
| U_{f}CₘGₘA_{d}T_{id}T | |
| SEAP-1116-m/f(as): | |
| 5′ PO₄-_{f}U_{f}CₘGₘAₘA_{f}CₘA_{f}UₘGₘA_{f}U_{f}CₘG_{f}U_{f}C_{f} | (SEQ ID 6) |
| U_{f}CₘAₘG_{d}T_{id}T | |
| SEAP-1116-f oligonucleotides: | |
| SEAP-1116-f(s): | |
| 5′ PO₄-_{f}C_{f}U_{f}G_{f}A_{f}G_{f}A_{f}C_{f}G_{f}A_{f}U_{f}C_{f}A_{f}U_{f}G_{f}U_{f} | (SEQ ID 5) |
| U_{f}C_{f}G_{f}A_{d}T_{id}T | |
| SEAP-1116-f(as): | |
| 5′ PO₄-_{f}U_{f}C_{f}G_{f}A_{f}A_{f}C_{f}A_{f}U_{f}G_{f}A_{f}U_{f}C_{f}G_{f}U_{f}C_{f} | (SEQ ID 6) |
| U_{f}C_{f}A_{f}G_{d}T_{id}T | |

C) SEAP-1296: Target sequence is position 1215-1235 of the SEAP open reading frame:

| | |
|---|---|
| CACGGTCCTCCTATACGGAAA | (SEQ ID 7) |

Unmodified SEAP-1296 RNA oligonucleotides (SEAP-1296-U oligonucleotides):

| | |
|---|---|
| SEAP-1296-U(s): | |
| 5′ PO₄-CGGUCCUCCUAUACGGAAAdTdT | (SEQ ID 8) |
| SEAP-1296-U(as): | |
| 5′ PO₄-UUUCCGUAUAGGAGGACCGdTdT | (SEQ ID 9) |

Modified SEAP-1296 RNA oligonucleotides (SEAP-1296-m/f oligonucleotides):

| | |
|---|---|
| SEAP-1296-m/f(s): | |
| 5′ PO₄-_{f}CₘGₘG_{f}U_{f}C_{f}C_{f}U_{f}C_{f}C_{f}UₘA_{f}UₘA_{f}CₘGₘ | (SEQ ID 8) |
| GₘAₘAₘA_{d}T_{id}T | |
| SEAP-1296-m/f(as): | |
| 5′ PO₄-_{f}U_{f}U_{f}U_{f}C_{f}CₘG_{f}UₘA_{f}UₘAₘGₘGₘAₘGₘGₘ | (SEQ ID 9) |
| A_{f}C_{f}CₘG_{d}T_{id}T | |

SEAP-1296-f oligonucleotides:

| | |
|---|---|
| SEAP-1296-f(s): | |
| 5′ PO₄-_{f}C_{f}G_{f}G_{f}U_{f}C_{f}C_{f}U_{f}C_{f}C_{f}U_{f}A_{f}U_{f}A_{f}C_{f}G_{f} | (SEQ ID 8) |
| G_{f}A_{f}A_{f}A_{d}T_{id}T | |
| SEAP-1296-_{f}(as): | |
| 5′ PO₄-_{f}U_{f}U_{f}U_{f}C_{f}C_{f}G_{f}U_{f}A_{f}U_{f}G_{f}G_{f}A_{f}G_{f}G_{f} | (SEQ ID 9) |
| A_{f}C_{f}C_{f}G_{d}T_{id}T | |

D) GL3-153: Target sequence is position 153-173 of the GL3 open reading frame

| | |
|---|---|
| CACTTACGCTGAGTACTTCGA | (SEQ ID 10) |

GL3-153 RNA oligonucleotides:

| | |
|---|---|
| GL3-153-U(s): | |
| 5′ PO₄-CUUACGCUGAGUACUUCGAdTdT | (SEQ ID 11) |
| GL-3-153-U(as): | |
| 5′ PO₄-UCGAAGUACUCAGCGUAAGdTdT | (SEQ ID 12) |

Nucleotide Modifications Key:
C=ribocytosine; A=riboadenonsine; U=ribouridine; G=riboguanosine; and, T=thymidine.

s=sense strand; as=antisense strand

m=—OCH₃ group at the ribose 2′ position

f=—F atom at the ribose 2′ position

d=—H atom at the ribose 2′ position (deoxy nucleotide)

i=nucleotide linked to its adjacent nucleotide through a 3′-PO₄-3′ linkage.

Preparation of siRNAs:

Sense and antisense oligonucleotides for each target sequence were annealed by mixing equimolar amounts of each and heating to 94° C. for 5 min, cooling to 90° C. for 3 min, then decreasing the temperature in 0.3° C. steps 250 times, holding at each step for 3 min. The resulting oligonucleotide duplex for GL3 was used as a control in the examples. The m/f siRNA oligonucleotides contain 2′-OCH₃ groups on pyrimidines, 2′-F groups on purines, and two thymidine deoxy nucleotides at the 3′ ends linked by an inverted linkage (3′-PO₄-3′). Combinations included both strands unmodified, sense strand only modified, anti-sense strand only modified, and both strands modified.

Naming convention for duplexed oligonucleotides is “name of sense strand oligonucleotide”:“name of antisense strand nucleotide”.

2. Injection of polynucleotides into mice and measurement of SEAP Activity. 4-6 week old mice (C57B1/6) were injected in the tail vein with a volume of Ringer's solution equaling 10% of the mouse's body weight and containing 1 μg pMIR141 (SEAP gene), 30 μg pMIR174 (expression vector containing the human Factor IX gene under control of the mouse albumin and AFP enhancer/promoter and human albumin 3′UTR) and the duplex oligonucleotide (5 μg). The entire injection volume was delivered in 5-7 seconds. For these studies, n=5. Mice were bled on the indicated days and the amount of SEAP activity in the plasma was measured using a commercially available kit according to the manufacturer's instructions (Tropix). The average SEAP expression was calculated for each group of mice. The data were normalized to the SEAP activity in animals that received the GL3-153-U(s):GL3-153-U(as) control duplex oligonucleotide.

3. Activity of duplex oligonucleotides containing 2′-OCH₃, 2′-F and 3′-idT substitutions in vivo. Duplex oligonucleotides targeting SEAP were prepared by annealing single strand oligonucleotides containing RNA bases with the complementary strand containing either RNA bases, or bases with 2′-OCH₃ substitutions on pyrmidines and 2′-F substitutions on purines. These combinations result in a set of four duplex oligonucleotides for each of the three target sequences in the SEAP gene. Each of the four duplexes for each target gene was injected separately into mice together with the SEAP expression plasmid. A duplex oligonucleotide targeting GL-3 was injected with the SEAP expression plasmid as a control. The level of SEAP activity in the blood of the injected animals was measured at different time points after injection. The results obtained using the SEAP-360 as the target sequence is shown in Table 1.

Injection of the duplex oligonucleotide without 2′-OCH₃and 2′-F substitutions, SEAP-360-U(s):SEAP-360-U(as) showed high knockdown activity at Day 1 after injection. SEAP levels in the blood of these mice were on average 0.04 of that of mice injected with the control duplex oligonucleotide GL3-153-U(s):GL3-153-U(as). The activity decreased over a number of days and SEAP level recovered to near or above those of the control by Day 10 after injection. Injection of the duplex oligonucleotide containing 2′-OCH₃ and 2′-F substitutions on both strands, SEAP-360-m/f(s):SEAP-360-m/f(as), resulted in inhibition of SEAP expression to levels similar to that of SEAP-360-U(s):SEAP-360-U(as) on Day 1 after injection. Activity of SEAP-360-m/f(s):SEAP-360-m/f(as) declined gradually and SEAP expression neared control levels by Day 15. Thus the longevity of activity was increased compared to SEAP-360-U(s):SEAP-360-U(as). Injection of the duplex oligonucleotide containing 2′-OCH₃ and 2′-F substitutions on the antisense strand only, SEAP-360-U(s):SEAP-360-m/f(as), resulted in higher inhibition of SEAP expression relative to that in mice receiving unmodified siRNA on Day 1 after injection. SEAP-360-U(s):SEAP-360-m/f(as) retained activity longer and SEAP expression did not reach near control levels until Day 17. Thus the longevity of knockdown activity increased compared to unmodified siRNA. Injection of the duplex oligonucleotide containing 2′-OCH₃ and 2′-F substitutions on the sense strand only, SEAP-360-m/f(s):SEAP-360-U(as), resulted in higher levels of inhibition of SEAP expression than in mice than SEAP-360-U(s):SEAP-360-U(as) on Day 1 after injection. This knockdown activity was maintained through Day 17. Activity of SEAP-360-m/f(s):SEAP-360-U(as) did not decay to near control levels until Day 35. Thus the longevity of activity was greatly increased compared to SEAP-360-U(s):SEAP-360-U(as). These data indicate that the duplexed oligonucleotide targeting the SEAP-360 sequence and containing a modified sense strand and an unmodified antisense strand possesses greater target gene knockdown activity and longevity than duplex oligonucleotides containing unmodified strands, modified antisense strand only, or modified sense and antisense strands.

TABLE 1
In vivo activity of SEAP-360 duplex oligonucleotides
containing 2′-OCH₃ and 2′-F substitutions

| | Duplex Oligonucleotide | | | | |
|---|---|---|---|---|---|
| | GL3-153U(s):GL3- | SEAP-360-U(s):SEAP- | SEAP-360-m/f(s):SEAP- | SEAP-360-U(s):SEAP- | SEAP-360-m/f(s):SEAP- |
| Day | 153-U(as) | 360-U(as) | 360-m/f(as) | 360-m/f(as) | 360-U(as) |
| 1 | 1.00 | 0.04 | 0.04 | 0.01 | 0.01 |
| 3 | 1.00 | 0.07 | 0.01 | 0.00 | 0.00 |
| 6 | 1.00 | 0.32 | 0.01 | 0.07 | 0.00 |
| 8 | 1.00 | 0.56 | 0.04 | 0.19 | 0.00 |
| 10 | 1.00 | 1.28 | 0.35 | 0.51 | 0.00 |
| 13 | 1.00 | 2.03 | 0.86 | 0.73 | 0.00 |
| 15 | 1.00 | 2.14 | 1.14 | 0.67 | 0.01 |
| 17 | 1.00 | 2.58 | 1.45 | 0.99 | 0.01 |
| 20 | 1.00 | 2.34 | 1.47 | 0.92 | 0.05 |
| 29 | 1.00 | 2.08 | 1.42 | 0.89 | 0.06 |
| 35 | 1.00 | 1.92 | 1.50 | 1.05 | 0.76 |
| 49 | 1.00 | 2.12 | 1.20 | 0.94 | 1.21 |

In order to test if modification of the sense strand leads to increased activity and longevity independent of nucleotide sequence, the experiment described above was repeated with duplex oligonucleotides targeting other sequences within the SEAP gene. The results obtained after injection of duplexes targeting the SEAP-1116 sequence are shown in Table 2. Injection of the duplex oligonucleotide without 2′-OCH₃ and 2′-F substitutions, SEAP-1116-U(s):SEAP-1116-U(as) showed moderate knockdown activity at Day 1 after injection. SEAP levels in the blood of these mice were on average 0.35 of that of mice injected with the control duplex oligonucleotide GL3-153-U(s):GL3-153-U(as). However, maximal activity was not observed until Day 3 at which time SEAP activity had decreased to 0.30 relative to that in mice receiving the control GL3-153-U(s):GL3-153-U(as) duplex oligonucleotide. The knockdown activity decreased over a number of days and SEAP levels recovered to near that of the control mice by Day 6 after injection. Injection of the duplex oligonucleotide containing 2′-OCH₃ and 2′-F substitutions on both strands, SEAP-1116-m/f(s):SEAP-1116-m/f(as), resulted in a lack of SEAP inhibition at all time points after injection, implying that the presence of these substituted nucleotides on both strands inactivated the duplex. Injection of the duplex oligonucleotide containing 2′-OCH₃ and 2′-F substitutions on the antisense strand only, SEAP-1116-U(s):SEAP-1116-m/f(as), resulted in slightly higher inhibition of SEAP expression than that achieved with SEAP-1116-U(s):SEAP-1116-U(as) on Day 1 after injection but inhibition is lost at the same rate and SEAP expression reached control levels by Day 6 after injection. Injection of the duplex oligonucleotide containing 2′-OCH₃ and 2′-F substitutions on the sense strand only, SEAP-1116-m/f(s):SEAP-1116-U(as), did not result in inhibition of SEAP expression until Day 3, at which time inhibition was near levels observed with the unmodified duplex SEAP-1116-U(s):SEAP-1116-U(as). Activity of SEAP-1116-m/f(s):SEAP-1116-U(as) then declined gradually and SEAP expression was near control levels by Day 10. Thus, the maximal activity and the longevity of activity of SEAP-1116-m/f(s):SEAP-1116-U(as) was similar to that of unmodified SEAP-1116-U(s):SEAP-1116-U(as).

TABLE 2
In vivo activity of SEAP-1116 duplex oligonucleotides
containing 2′-OCH₃ and 2′-F substitutions in vivo

| | Duplex Oligonucleotide | | | | |
|---|---|---|---|---|---|
| | GL3-153U(s):GL3- | SEAP-1116-U(s):SEAP- | SEAP-1116-m/f(s):SEAP- | SEAP-1116-U(s):SEAP- | SEAP-1116-m/f(s):SEAP- |
| Day | 153-U(as) | 1116-U(as) | 1116-m/f(as) | 1116-m/f(as) | 1116-U(as) |
| 1 | 1.00 | 0.35 | 2.59 | 0.10 | 1.62 |
| 3 | 1.00 | 0.30 | 1.28 | 0.37 | 0.40 |
| 6 | 1.00 | 1.21 | 1.33 | 1.29 | 0.59 |
| 8 | 1.00 | 1.97 | 1.42 | 1.39 | 0.73 |
| 10 | 1.00 | 1.94 | 1.15 | 1.35 | 1.04 |
| 13 | 1.00 | 1.82 | 1.34 | 1.24 | 1.51 |
| 15 | 1.00 | 1.82 | 1.24 | 1.16 | 1.79 |
| 27 | 1.00 | 1.72 | 1.16 | 1.19 | 1.31 |

The results obtained after injection of duplexes targeting the SEAP-1296 sequence are shown in Table 3. Injection of the duplex oligonucleotide without 2′-OCH₃ and 2′-F substitutions targeting the SEAP-1296 sequence, SEAP-1296-U(s):SEAP-1296-U(as) showed moderate knockdown activity at Day 1 after injection. SEAP levels in the blood of these mice were on average 0.46 of that of mice injected with the control duplex oligonucleotide GL3-153-U(s):GL3-153-U(as). The knockdown activity decreased over a number of days and SEAP expression recovered to near that in mice receiving the control GL3-153-U(s):GL3-153-U(as) by Day 13 after injection. Injection of the duplex oligonucleotide containing 2′-OCH₃ and 2′-F substitutions on both strands, SEAP-1296-m/f(s):SEAP-1296-m/f(as), resulted in moderate knockdown activity. However, peak knockdown activity was not observed until Day 8 after injection and at this time point SEAP expression was only inhibited to 0.50 of that in mice receiving the control GL3-153-U(s):GL3-153-U(as) duplex oligonucleotide. The knockdown activity decreased over time and SEAP expression recovered to near that of the control mice by Day 13 after injection. Injection of the duplex oligonucleotide containing 2′-OCH₃ and 2′-F substitutions on the antisense strand only, SEAP-1116-U(s):SEAP-1116-m/f(as), resulted in a lack of SEAP inhibition at all time points after injection, indicating that the presence of the substitutions in the antisense strand when duplexed with unsubstituted sense strand inactivated the duplex for target gene knockdown. Injection of the duplex oligonucleotide containing 2′-OCH₃ and 2′-F substitutions on the sense strand only, SEAP-1296-m/f(s):SEAP-11296-U(as), resulted in inhibition of SEAP expression that was greater than that of SEAP-1296-U(s):SEAP-1296-U(as) on Day 1 after injection. At subsequent time points, knockdown increased to a maximum at Day 8, at which time SEAP expression was just 0.02 of that observed in the control group which received GL3-153-U(s):GL3-153-U(as). Activity of SEAP-1296-m/f(s):SEAP-1296-U(as) persisted until about Day 24. Thus the maximal knockdown achieved and the longevity of activity was greatly increased compared to SEAP-1296-U(s):SEAP-1296-U(as). These data indicate that the duplexed oligonucleotide targeting the SEAP-1296 sequence and containing a modified sense strand and an unmodified antisense strand possessed greater target gene knockdown activity and longevity than duplexes containing unmodified strands, modified antisense strand only, or modified sense and antisense strands.

TABLE 3
In vivo activity of SEAP-1296 duplex oligonucleotides containing 2′-OCH₃ and
2′-F substitutions in vivo

| | Duplex Oligonucleotide | | | | |
|---|---|---|---|---|---|
| | GL3-153U(s):GL3- | SEAP-1296-U(s):SEAP- | SEAP-1296-m/f(s):SEAP- | SEAP-1296-U(s):SEAP- | SEAP-1296-m/f(s):SEAP- |
| Day | 153-U(as) | 1296-U(as) | 1296-m/f(as) | 1296-m/f(as) | 1296-U(as) |
| 1 | 1.00 | 0.46 | 0.89 | 2.21 | 0.37 |
| 3 | 1.00 | 0.46 | 0.67 | 1.79 | 0.04 |
| 6 | 1.00 | 0.56 | 0.61 | 2.81 | 0.03 |
| 8 | 1.00 | 0.73 | 0.50 | 1.55 | 0.02 |
| 10 | 1.00 | 0.71 | 0.56 | 1.25 | 0.05 |
| 13 | 1.00 | 0.86 | 0.85 | 1.51 | 0.29 |
| 15 | 1.00 | 0.84 | 0.87 | 1.48 | 0.42 |
| 17 | 1.00 | 1.67 | 1.02 | 1.98 | 0.56 |
| 20 | 1.00 | 1.46 | 0.89 | 1.49 | 0.68 |
| 24 | 1.00 | 1.58 | 1.53 | 2.08 | 1.42 |
| 31 | 1.00 | 1.24 | 1.57 | 2.45 | 1.95 |

Comparing the results obtained for the three duplex oligonucleotides targeting three different sequences in the SEAP gene, it is concluded that two of three duplexes containing 2′-OCH₃ and 2′-F substitutions and _{id}T at the 3′ end on the sense strand have greater knockdown activity and longevity of activity than duplexes containing unmodified strands, modified antisense strand only, or modified sense and antisense strands. The third duplex, that targeting SEAP-1116, had similar activity to the unmodified duplex. Thus, modification of the sense strand of the duplex oligonucleotide generally improves knockdown activity and the longevity of knockdown.

4. Activity of duplex oligonucleotides containing 2′-F and 3′_{id}T substitutions in vivo. Duplex oligonucleotides targeting SEAP were prepared by annealing single strand oligonucleotides containing RNA bases with the complementary strand containing either RNA nucleotides, or bases with nucleotides containing 2′-F substitutions. This results in a set of four duplex oligonucleotides for each of the three target sequences in the SEAP gene. Each of the four duplexes for each target gene was injected separately into mice together with the SEAP expression plasmid. A duplex oligonucleotide targeting GL-3, GL3-153-U(s):GL3-153-U(as) was injected with the SEAP expression plasmid as a control. The level of SEAP activity in the blood of the injected animals was measured at different time points after injection.

The results obtained using the SEAP-360 as the target sequence are shown in Table 4. Injection of the duplex 2′-F substitutions, SEAP-360-U(s):SEAP-360-U(as) showed high knockdown activity at Day 1 after injection. SEAP levels in the blood of these mice were on average 0.02 of that of mice injected with the control duplex oligonucleotide GL3-153-U(s):GL3-153-U(as). The activity decreased over a number of days and SEAP level recovered to near or above those of the control by Day 20 after injection. Injection of the duplex oligonucleotide containing 2′-F substitutions on both strands, SEAP-360-f(s):SEAP-360-f(as), results in inhibition of SEAP expression to levels near that in mice injected SEAP-360-U(s):SEAP-360-U(as) on Day 3 after injection. Unlike mice injected with SEAP-360-U(s):SEAP-360-U(as), maximal activity is not achieved until Day 3. Activity of SEAP-360-f(s):SEAP-360-f(as) declined gradually and reached a plateau at Day 15 through the end of the experiment. Injection of the duplex oligonucleotide containing 2′-F substitutions on the antisense strand only, SEAP-360-U(s):SEAP-360-f(as), resulted in slightly lower inhibition of SEAP expression than that in mice injected with SEAP-360-U(s):SEAP-360-U(as) on Day 1 after injection. Activity of SEAP-360-U(s):SEAP-360-f(as) declined over time and then plateaued at Day 10 until the end of the experiment. Injection of the duplex oligonucleotide containing 2′-F substitutions on the sense strand only, SEAP-360-f(s):SEAP-360-U(as), resulted in greater inhibition of SEAP expression than that achieved in mice injected with SEAP-360-U(s):SEAP-360-U(as) on Day 7. Unlike mice injected with SEAP-360-U(s):SEAP-360-U(as) in which maximal activity is attained by Day 1, maximal activity of SEAP-360-f(s):SEAP-360-U(as) is not achieved until Day 7. Activity of SEAP-360-f(s):SEAP-360-U(as) then declined gradually and SEAP expression returned to near control levels by Day 20. Thus the knockdown activity of SEAP-360-f(s):SEAP-360-U(as) was increased compared to SEAP-360-U(s):SEAP-360-U(as).

TABLE 4
In vivo activity of SEAP-360 duplex oligonucleotides containing 2′-F substitutions
at all base paired positions

| | Duplex Oligonucleotide | | | | |
|---|---|---|---|---|---|
| | GL3-153U(s):GL3- | SEAP-360-U(s):SEAP- | SEAP-360-f(s):SEAP- | SEAP-360-U(s):SEAP- | SEAP-360-f(s):SEAP- |
| Day | 153-U(as) | 360-U(as) | 360-f(as) | 360-f(as) | 360-U(as) |
| 1 | 1.00 | 0.02 | 0.19 | 0.04 | 0.31 |
| 3 | 1.00 | 0.02 | 0.09 | 0.08 | 0.04 |
| 7 | 1.00 | 0.09 | 0.24 | 0.48 | 0.01 |
| 10 | 1.00 | 0.33 | 0.41 | 0.71 | 0.05 |
| 13 | 1.00 | 0.51 | 0.48 | 0.69 | 0.10 |
| 15 | 1.00 | 0.69 | 0.55 | 0.75 | 0.34 |
| 17 | 1.00 | 0.59 | 0.51 | 0.69 | 0.47 |
| 20 | 1.00 | 1.08 | 0.78 | 0.91 | 1.21 |
| 24 | 1.00 | 0.99 | 0.58 | 0.77 | 1.57 |

The results obtained after injection of duplexes targeting the SEAP-1116 sequence are shown in Table 5. Injection of the duplex oligonucleotide without 2′-F substitutions, SEAP-1116-U(s):SEAP-1116-U(as) showed high knockdown activity at Day 1 after injection. SEAP levels in the blood of these mice were on average 0.16 of that of mice injected with the control duplex oligonucleotide GL3-153-U(s):GL3-153-U(as). The knockdown activity decreased over a number of days and SEAP expression recovered to near that in the control mice injected with GL3-153-U(s):GL3-153-U(as) by Day 8 after injection. Injection of the duplex oligonucleotide containing 2′-F substitutions on both strands, SEAP-1116-f(s):SEAP-1116-f(as), resulted in slightly higher inhibition of SEAP expression than in mice injected with SEAP-1116-U(s):SEAP-1116-U(as), with maximal inhibition on Day 3 after injection. SEAP levels in the blood of these mice were on average 0.06 of that of mice injected with the control duplex oligonucleotide GL3-153-U(s):GL3-153-U(as). The knockdown activity decreased over time and SEAP expression recovered to near that of mice receiving the control duplex oligonucleotide by Day 15 after injection. Injection of the duplex oligonucleotide containing 2′-F substitutions on the antisense strand only, SEAP-1116-U(s):SEAP-1116-f(as), resulted in slightly higher inhibition of SEAP expression than that achieved with SEAP-1116-U(s):SEAP-1116-U(as) on Day 1 after injection but SEAP expression reached near control levels by Day 8. Injection of the duplex oligonucleotide containing 2′-F substitutions on the sense strand only, SEAP-1116-f(s):SEAP-1116-U(as), resulted in the highest level of inhibition of all four duplexes targeting the SEAP-1116 sequence, reaching maximum inhibition at Day 3 after injection. The knockdown activity slowly decreased over time and SEAP expression did not recover to near that in mice receiving the control duplex oligonucleotide until Day 21 after injection. Thus the knockdown activity and longevity of knockdown is greatly increased compared to SEAP-1116-U(s):SEAP-1116-U(as).

TABLE 5
In vivo activity of SEAP-1116 duplex oligonucleotides containing
2′-F substitutions at all base paired positions

| | Duplex Oligonucleotide | | | | |
|---|---|---|---|---|---|
| | GL3-153U(s): GL3- | SEAP-1116-U(s):SEAP- | SEAP-1116-f(s):SEAP- | SEAP-1116-U(s):SEAP- | SEAP-1116-f(s):SEAP- |
| Day | 153-U(as) | 1116-U(as) | 1116-f(as) | 1116-f(as) | 1116-U(as) |
| 1 | 1.00 | 0.16 | 0.13 | 0.08 | 0.07 |
| 3 | 1.00 | 0.24 | 0.06 | 0.16 | 0.06 |
| 6 | 1.00 | 0.48 | 0.19 | 0.65 | 0.13 |
| 8 | 1.00 | 0.92 | 0.52 | 0.93 | 0.34 |
| 10 | 1.00 | 0.99 | 0.60 | 0.88 | 0.41 |
| 13 | 1.00 | 1.25 | 0.75 | 0.88 | 0.71 |
| 15 | 1.00 | 1.25 | 0.91 | 0.89 | 0.68 |
| 17 | 1.00 | 1.60 | 0.99 | 1.12 | 0.88 |
| 21 | 1.00 | 1.52 | 1.08 | 1.09 | 0.90 |

The results obtained after injection of duplexes targeting the SEAP-1296 sequence are shown in Table 3. Injection of the duplex oligonucleotide without 2′-F substitutions targeting the SEAP-1296 sequence, SEAP-1296-U(s):SEAP-1296-U(as) showed knockdown activity beginning at Day 1 after injection. SEAP levels in the blood of these mice were on average 0.10 of that of mice injected with the control duplex oligonucleotide GL3-153-U(s):GL3-153-U(as) on Day 1 after injection. The knockdown activity decreased over time and SEAP expression recovered to near that in mice receiving the control duplex oligonucleotide by Day 10 after injection. Injection of the duplex oligonucleotide containing 2′-F substitutions on both strands, SEAP-1296-f(s):SEAP-1296-f(as), resulted in knockdown activity comparable to that obtained in mice injected with SEAP-1296-U(s):SEAP-1296-U(as). However, peak knockdown activity was not observed until Day 3 after injection. The knockdown activity decreased over time from that point and plateaued by Day 15. Injection of the duplex oligonucleotide containing 2′-F substitutions on the antisense strand only, SEAP-1296-U(s):SEAP-1296-f(as), resulted in inhibition of SEAP expression to a similar degree as observe in mice receiving the unmodified duplex oligonucleotide, SEAP-1296-U(s):SEAP-1296-U(as), at all time points after injection. Injection of the duplex oligonucleotide containing 2′-F substitutions on the sense strand only, SEAP-1296-f(s):SEAP-1126-U(as), resulted in inhibition of SEAP expression on Day 3 that was greater than that observed for all other duplex oligonucleotides targeting SEAP-1296. Activity of SEAP-1296-m/f(s):SEAP-1296-U(as) then declined gradually and SEAP expression recovered to near control levels by Day 17 after injection. Thus the maximal knockdown achieved and the longevity of knockdown was greatly increased compared to SEAP-1296-U(s):SEAP-1296-U(as). These data indicate that the duplexed oligonucleotide targeting the SEAP-1296 sequence and containing a modified sense strand and an unmodified antisense strand possesses greater target gene knockdown activity and longevity than duplexes containing unmodified strands, modified antisense strand only, or modified sense and antisense strands.

TABLE 6
In vivo activity of SEAP-1296 duplex oligonucleotides containing
2′-F substitutions at all base paired positions

| | Duplex Oligonucleotide | | | | |
|---|---|---|---|---|---|
| | GL3-153U(s):GL3- | SEAP-1296-U(s):SEAP- | SEAP-1296-f(s):SEAP- | SEAP-1296-U(s):SEAP- | SEAP-1296-f(s):SEAP- |
| Day | 153-U(as) | 1296-U(as) | 1296-f(as) | 1296-f(as) | 1296-U(as) |
| 1 | 1.00 | 0.10 | 0.16 | 0.08 | 0.10 |
| 3 | 1.00 | 0.18 | 0.12 | 0.27 | 0.03 |
| 7 | 1.00 | 0.59 | 0.20 | 0.84 | 0.07 |
| 10 | 1.00 | 0.91 | 0.34 | 1.26 | 0.25 |
| 13 | 1.00 | 1.03 | 0.47 | 0.99 | 0.37 |
| 15 | 1.00 | 1.26 | 0.58 | 1.19 | 0.70 |
| 17 | 1.00 | 1.47 | 0.70 | 1.13 | 1.04 |
| 20 | 1.00 | 1.52 | 0.48 | 1.15 | 1.22 |
| 24 | 1.00 | 1.21 | 0.39 | 0.90 | 1.27 |

Comparing the results obtained for the three duplex oligonucleotides targeting three different sequences in the SEAP gene, it is concluded that all three duplexes containing 2′-F substitutions at all base paired positions on the sense strand only and idT at the 3′ end have greater knockdown activity and greater than or equal to longevity of activity than duplexes containing unmodified strands, modified antisense strand only, or modified sense and antisense strands.

The foregoing is considered as illustrative only of the principles of the invention.

Furthermore, since numerous modifications and changes will readily occur to those skilled in the art, it is not desired to limit the invention to the exact construction and operation shown and described. Therefore, all suitable modifications and equivalents fall within the scope of the invention.

We claim:

1. A compound for inhibiting expression of a gene in a mammalian cell comprising: a double strand oligonucleotide consisting of a sequence that is substantially complementary to said gene and having a least one strand of said double strand oligonucleotide consisting essentially of 2′-methoxy purine ribonucleotides and 2′-fluoro pyrimidine ribonucleotides.

2. The double strand oligonucleotide of claim 1 wherein said strand consists of a sense strand.

3. The double strand oligonucleotide of claim 1 wherein said strand consists of an antisense strand.

4. The double strand oligonucleotide of 1 wherein both strands consist essentially of 2′-methoxy purine ribonucleotides and 2′-fluoro pyrimidine ribonucleotides.

5. The compound of claims 1 wherein the oligonucleotide further comprises at least one 3′ overhang.

6. The compound of claim 5 wherein the 3′ overhang contains a terminal inverted nucleotide.

7. The compound of claim 5 wherein the 3′ overhang contains a terminal inverted abasic nucleotide.

8. The compound of claim 1 wherein said double strand oligonucleotide comprises two annealed substantially complementary oligonucleotides.

9. The compound of claim 1 wherein said double strand oligonucleotide comprises a hairpin oligonucleotide.

10. A compound for inhibiting expression of a gene in a mammalian cell comprising: a double strand oligonucleotide consisting of a sequence that is substantially complementary to said gene and having a least one strand of said double strand oligonucleotide consisting essentially of 2′-fluoro ribonucleotides.

11. The double strand oligonucleotide of claim 10 wherein said strand consists of a sense strand.

12. The double strand oligonucleotide of claim 10 wherein said strand consists of an antisense strand.

13. The double strand oligonucleotide of 10 wherein both strands consist essentially of 2′-fluoro pyrimidine ribonucleotides.

14. The compound of claims 10 wherein the oligonucleotide further comprises at least one 3′ overhang.

15. The compound of claim 14 wherein the 3′ overhang contains a terminal inverted nucleotide.

16. The compound of claim 14 wherein the 3′ overhang contains a terminal inverted abasic nucleotide.

17. The compound of claim 10 wherein said double strand oligonucleotide comprises two annealed substantially complementary oligonucleotides.

18. The compound of claim 10 wherein said double strand oligonucleotide comprises a hairpin oligonucleotide.


SEQUENCE LISTING (12 sequences)

SEQ ID NO: 1 - DNA, 21 bp, Homo sapiens
caagggcaacttccagaccat

SEQ ID NO: 2 - DNA, 21 bp, Homo sapiens
agggcaacuuccagaccautt

SEQ ID NO: 3 - DNA, 21 bp, Homo sapiens
auggucuggaaguugcccutt

SEQ ID NO: 4 - DNA, 21 bp, Homo sapiens
gactgagacgatcatgttcga

SEQ ID NO: 5 - DNA, 21 bp, Homo sapiens
cugagacgaucauguucgatt

SEQ ID NO: 6 - DNA, 21 bp, Homo sapiens
ucgaacaugaucgucucagtt

SEQ ID NO: 7 - DNA, 21 bp, Homo sapiens
cacggtcctcctatacggaaa

SEQ ID NO: 8 - DNA, 21 bp, Homo sapiens
cgguccuccuauacggaaatt

SEQ ID NO: 9 - DNA, 21 bp, Homo sapiens
uuuccguauaggaggaccgtt

SEQ ID NO: 10 - DNA, 21 bp, Photinus pyralis
cacttacgctgagtacttcga

SEQ ID NO: 11 - DNA, 21 bp, Photinus pyralis
cuuacgcugaguacuucgatt

SEQ ID NO: 12 - DNA, 21 bp, Photinus pyralis
ucgaaguacucagcguaagtt